(19) United States
(12) Patent Application Publication
(10) Pub. No.: US 2004/0082817 A1
(43) Pub. Date: Apr. 29, 2004

(54) Method of phenol and carbonyl production

(75) Inventor: Olga Tarasovna Kasaikina, Moscow (RU)

Correspondence Address: HOGAN & HARTSON LLP, 555 13th Street, N.W., Washington, DC, 20004, US

(73) Assignee: NEUROK LLC

(21) Appl. No.: 10/279,012

(22) Filed: Oct. 24, 2002

(51) Int. Cl. C07C 37/08; C07C 45/53

(52) U.S. Cl. 568/385; 568/798

(57) ABSTRACT

The present invention describes a high yield and simplified method of preparing phenols and carbonyl compounds, such as acetaldehyde, through the catalytic decomposition of aromatic hydroperoxides. The synthetic process generally consists of decomposing hydroperoxides under a positive gas atmosphere and at an elevated temperature (80 to 100° C.) in the presence of a catalytic amount (0.01-0.8 mass percent (%)) of an anionic surface-active agent of the formula R—OSO₃M or R—OP₃Z₂, where “M” is Na or K, where “Z” is Na, K or an alkyl groups containing between ten and fourteen carbons (C₁₀-C₁₄) and where “R” is an alkyl group consisting of between ten and fourteen carbons (C₁₀-C₁₄).

### BACKGROUND OF THE INVENTION

1. Field of the Invention

The present invention relates to a method of preparing phenols and carbonyl compounds, such as acetaldehyde, by catalytic decomposition of aromatic hydroperoxides.

2. Discussion of the Related Art

Phenol (C₆H₅OH) has diverse applications as a raw product for the production of phenolics (phenol aldehyde resin, resol resin), ε-caprolactam, adipinic acid, synthetic polyamides, and for the production of synthetic fibers (caprone, nylon), plastics, aniline, bisphenols, alkylphenols, pesticides and plasticizing agents. Phenol is also applied in the synthesis of disinfectants and pharmaceuticals such as aspirin, salol, phenolphtalein, etc. In the petrochemical industry, phenol is useful in selective oil purification systems and for various chemical analyses.

Carbonyl compounds, such as acetaldehyde (CH₃CHO), are used in the production of, among other things, cellulose, acetates, peracetic acids, acetic anhydride, ethylacetate, alkylamines, pentaerythrite, and alkylpyridines.

A number of methods are known for the production of phenols and carbonyl compounds. These methods can generally be classified as either acid catalyzed or non-acid catalyzed.

Acid catalyzed methods dominate traditional large-scale commercial production of phenols and carbonyl compounds employing some form of heterogeneous acid catalyzed process. Typically, sulfuric acid (H₂SO₄) is the acid catalyst used.

For example, the Cumene Method (or Oudris-Sergeev Method) involves the decomposition of cumene hydroperoxide in the presence of an acidic catalyst, such as sulfuric acid. With reported yields of about 96%, this method accounts for more than 90% of commercially produced phenol.

The Sulfurization Method entails sulfonating benzene by sulfuric acid at an elevated temperature (120°-150° C.) and pressure (0.25 MPascal) followed by alkaline melting of the formed sulfoaromatic acid with NaOH at 320° C. This method is not as popular as the Cumene Method, and produces a yield of about 92%.

Both the Cumene Method and Sulfurization Method suffer from a variety of failings. These shortcomings include: resinification, phenolic acid formation and acid neutralization costs and wastes. For an article discussing these problems please see B. D. Kruzhalov and B. I. Golovanenko, “Joint Production of Phenol and Acetone,” Goskhimizdat, (Moscow, 1963; Russian).

An improved acid catalyzed system for the joint production of phenol and acetone has been described in Russian Federation Patent No. 2068404 (Oct. 27, 1996). The method involves a two-stage decomposition of isopropyl benzene/hydroperoxide mixture in the presence of sulfuric acid. Isopropyl benzene is added in about 18-25 mass percent of initial hydroperoxide concentration. Dilution of the initial hydroperoxide allows for an increase in the degree of decomposition and consequent increase in phenol output. The catalyst can easily be washed away from the decomposed reaction products with water. This separation, however, takes a considerable amount of time—up to 4 hours—therefore substantially increasing the duration of the method.

U.S. Pat. No. 4,246,203 (Jun. 20, 1981) describes an efficient method for the production of phenol and acetone from isopropyl benzene hydroperoxide. Instead of removing the reaction products via a liquid phase separation, heat is used to remove phenol and carbonyl component in the gas phase. This procedure eliminates the necessity to neutralize the reaction products. Volatile impurities are removed via flash pyrolysis. The disadvantage of this method is relatively low distillate yields (up to 91.6%) indicative of an insufficient degree of hydroperoxide decomposition, as well as a moderate yield of phenol and acetone (up to 91.7 mol. percent and 93.2 mol. percent, respectively).

Non-acid catalyzed methods have been developed as an alternative to the acid catalyzed methods. Efforts seeking improvements over traditional acid catalyzed decompositions have focused on the use of heterogeneous organometallic catalysts. For example, Russian Federation Patent No. 2,039,593 (Jul. 20, 1995) discloses the production of phenol and acetone through the use of hydrated niobium pentaoxide NbO₅.nH₂O calcined at 100°-300° C. and/or niobium phosphate Nb₂O₅.nP₂O₅.nH₂O (m=0.5-5) calcined at 100°-150° C. These catalysts are very effective because they (1) have a high activity and selectivity, (2) they do not resinify reaction products, and (3) they can be easily separated from the reaction mass by filtration and/or sedimentation techniques. Unfortunately, their cost and inaccessibility limits the production scale use of these catalysts.

U.S. Pat. No. 4,262,153 describes the use of the carbamates, monothionates and dithionates of Zn, Ni, Fe, Co, Cu and Cd. These catalysts are used in quantities of about 0.1 to 5 mass percent of hydroperoxide and are especially efficient for the decomposition of tertiary arylhydroperoxides, e.g. cumene hydroperoxide and cyclohexyl benzene hydroperoxide. These types of decompositions are performed using inert organic solvents (benzene, toluene) over a wide temperature range (50°-200° C.). While efficient and fairly simple in design, use of organometallic catalysts and organic solvents makes these methods unsuitable for production scale use.

Other organometallic catalyzed methods are known as well, but suffer from similar shortcomings. For example, in the Toluene Method according to W. W. Kaeding, Petrol Refiner, 43 (11), 173 (1964), toluene is catalytically converted into benzoic acid in the presence of atmospheric oxygen at elevated temperatures (150°-170° C., 1.5 MPascals) using a cobalt catalyst. Once formed, the benzoic acid undergoes an oxidative decarboxylation at 230°-240° C. with a copper catalyst to form phenol. The overall yield is 82%.

The Chlorobenzene, which yields between 90 and 95%, requires the oxidative hydrochlorination of benzene at 270° C. with an iron or copper oxide catalyst. The formed chlorobenzene is then hydrolized with steam at 450°-550° C. in the presence of a silicon dioxide catalyst to yield the final product.

The Cyclohexane Method, described by A. I. Rakhimov, “Chemistry and Technology of Organic Peroxides,” M.J. Chemistry, 392 (1979), involves the oxidation of cyclohexane by atmospheric oxygen at elevated temperatures. (130°-160° C.) and pressure (3-4 MPascals) in the presence of a cobalt catalyst. The reaction produces a mixture of cyclohexanol and cyclohexanone requiring further catalytic (cobalt, platinum, nickel) dehydrogenation at 250-420° C. The overall yield is 82%.

For a more complete discussion of metal catalyzed oxidations see R. A. Sheldon and J. K. Kochi, “Metal-Catalyzed Oxidation of Organic Compounds in the Liquid Phase” Oxidation and Combustion Revs, Vol. 5, 135-242 (1973).

### SUMMARY OF THE INVENTION

The present invention is directed to a high yield and simplified method of preparing phenols and carbonyl compounds, such as acetaldehyde, by catalytic decomposition of aromatic hydroperoxides. The invention is applicable to the organic synthetic and petrochemical industries. The method generally consists of decomposing hydroperoxides under a positive gas atmosphere (air, nitrogen or carbon dioxide) and at an elevated temperature (80° to 100° C.) in the presence of a catalytic amount (0.01-0.8 mass percent (%)) of an anionic surface-active agent of the formula R—OSO₃M or R—OP₃Z₂, where “M” is Na or K, where “Z” is Na, K or an alkyl groups containing between ten and fourteen carbons (C₁₀-C₁₄) and where “R” is an alkyl group consisting of between ten and fourteen carbons (C₁₀-C₁₄). Examples include sodium dodecylsulfate (SDS), sodium decylsulfate or sodium tridecylsulfate. Additional features and advantages of the invention will be set forth in the description which follows, and, in part, will be apparent from the description, or may be learned by practice of the invention. The objectives and other advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### DETAILED DESCRIPTION OF THE PREFERRED EMBODIMENTS

The present invention is directed to a method of preparing phenols and carbonyl compounds, such as acetaldehyde, by catalytic decomposition of aromatic hydroperoxides. The objective of the invention is to provide a simplified high-yield synthetic method for the production of phenols and carbonyl compounds. A further objective of the invention is to exclude the use of sulfuric acid from the process by utilizing available and affordable heterogeneous catalysts.

The invention generally consists of decomposing hydroperoxides under a positive gas atmosphere (air, nitrogen or carbon dioxide) and at an elevated temperature (80°to 100° C.) in the presence of a catalytic amount (0.01-0.8 mass percent (%)) of an anionic surface-active agent of the formula R—OSO₃M or R—OP₃Z₂, where “M” is Na or K, where “Z” is Na, K or an alkyl groups containing between ten and fourteen carbons (C₁₀-C₁₄) and where “R” is an alkyl group consisting of between ten and fourteen carbons (C₁₀-C₁₄). Examples include sodium dodecylsulfate (SDS), sodium decylsulfate or sodium tridecylsulfate.

The process is especially appropriate for phenol and acetaldehyde production. Both can efficiently prepared with little or no waste by heterolytic decomposition of ethylbenzene hydroperoxide (EHP) using catalytic anionic surface-active agents. The reaction takes little time (20 to 40 minutes) and occurs at moderate temperatures (80° to 100° C.). No sulfuric acid is used in this process, thus no acid neutralization step is required. The starting material, ethylbenzene hydroperoxide, can be prepared by the catalytic oxidation of ethylbenzene.

### EXAMPLES

General Experimental Conditions: Initial hydroperoxide concentrations were approximately 2 mass percent. Anionic surface-active agent concentration was approximately 0.8 mass percent. Specific anionic surface-active agents used were: sodium dodecylsulfate (SDS), sodium decylsulfate or sodium tridecylsulfate. The reactions were performed at 100° C. for approximately 40 minutes. Ethylbenzene hydroperoxide (EHP) dissociation level was between 98.5 to 99% and phenol yield was between 90-98%.

EHP was produced by ethylbenzene oxidation with atmospheric oxygen at 120° C. in the presence of an alkali additive (NaOH) and stainless steel 12×18H10T shavings. Hydroperoxide concentration is 0.5 M (8%). It should be noted that the use of the alkali additive during EHP synthesis leads to a subsequent decrease in phenol production when SDS is used for hydroperoxide decomposition.

### Example 1

SDS (0.29 g) and oxidized ethylbenzene (100 ml) containing between 2 and 8 mass percent of hydroperoxide were combined in a round bottom flask equipped with a reverse condenser. The reaction was heated for 30 minutes between 90 and 120° C. and yielded between 90 and 95 mole % of phenol relative to decomposed hydroperoxide. When allowed to continue until full hydroperoxide decomposition, the reaction mixture turned brown and the coloration became more intense at higher temperatures.

The results of these reactions are detailed in the table below. Hydroperoxide and SDS concentration is given in mass percentage relative to the total mass of the reaction mixture. Effluent gases contain acetaldehyde (see example 3 below).

TABLE 1
Effect of temperature on the EHP decomposition rate and
phenol yield in the presence of SDS.

| | | | | | | Perox- | |
|---|---|---|---|---|---|---|---|
| | Experimental conditions | | | | | ide | |
| Experi- | | | EHP | SDS | Time | dissoci- | Phenol |
| ment | Temp. | | conc. | conc. | required* | ation | content** |
| No | (C.) | Gas | (%) | (%) | (mins) | (%) | (%) |
| 1 | 90 | N₂ | 6.2 | 0.33 | 90 | 99.6 | 89 |
| 2 | 110 | N₂ | 8.2 | 0.033 | 90 | 98.6 | 70 |
| 3 | 110 | N₂ | 8.2 | 0.33 | 20 | 99.9 | 90 |
| 4 | 120 | N₂ | 8.2 | 0.33 | 10 | 99.8 | 66 |
| 5 | 100 | N₂ | 8.2 | 0.33 | 30 | 99.9 | 90 |

*For 99% EHP decomposition;
**Relative to decomposed EHP.

TABLE 2
Effect of atmosphere changes on phenol yield on EHP
decomposition in the presence of SDS.*

| | Experimental conditions | | | | | Peroxide | |
|---|---|---|---|---|---|---|---|
| Experi- | | | EHP | SDS | Dura- | dissoci- | Phenol |
| ment | Temp. | | conc. | conc. | tion, | ation | content** |
| No | (C.) | Gas | (%) | (%) | (mins) | (%) | (%) |
| 6 | 100 | N₂ | 8.2 | 0.33 | 22 | 95.7 | 93 |
| 6′ | 100 | N₂ | 8.2 | 0.33 | 22 | 94.7 | 93 |
| 7 | 100 | air | 8.2 | 0.33 | 22 | 96.1 | 83 |
| 8 | 100 | CO₂ | 8.2 | 0.33 | 22 | 96.1 | 77 |

*Note.
Reaction product analysis was performed using gas chromatograph using “Tzvet 5” on a dinonylphthalate plated chromaton column.
**Relative to decomposed EHP.

### Example 2

Following the same experimental conditions described in Example 1 above, the effect on EHP decomposition using sodium tridecylsulfate (TDS), decylsulfate (DS), dedecylbenzenesulfonate (DBS), phosphoric acid (H₃PO₄) and an octadecanol/H₃PO₄/Sodium Hydroxide (HaOH) mixture were studied. The results of these experiments are detailed in Table 3.

TABLE 3
Effects of Different ASAA Catalysts

| | Experimental conditions | | | | | Perox- | |
|---|---|---|---|---|---|---|---|
| | | | | EHP | Dura- | ide | Phenol |
| | Temp. | | Additive | conc. | tion | decomp, | yield |
| Exp. | (C.) | Catalyst | conc. | (%) | (min) | (%) | (%) |
| 1 | 100 | TDS | 0.8% | 1.6 | 40 | 98 | 87 |
| 2 | 120 | DS | 0.8% | 1.6 | 30 | 99.8 | 99 |
| 3 | 120 | DBS | 0.1% | 1.6 | 120 | None | 0 |
| 4 | 100 | Octa- | 0.025 M | 1.6 | 50 | 51 | 84 |
| | | decanol | | | | | |
| | | H₃PO₄ | 0.025 M | | | | |
| | | NaOH | 0.05 M | | | | |
| 5 | 100 | H₃PO₄ | 0.025 M | 1.6 | 40 | 99.9 | 60 |

### Example 3

After completing the procedures described in Example 1, acetaldehyde was extracted from the reaction mixture as a component of the atmospheric gases in the reaction. The acetaldehyde was isolated in its natural form or immediately oxidized into acetic acid.

Isolation of pure acetaldehyde was accomplished by trapping the gases and volatile products formed in the ethylbenzene hydroperoxide decomposition process and flushing them through a 20% H₂SO₄ solution at 0° C. As a result, small white crystals of acetaldehyde tetramer or metaldehyde.

Direct conversion of acetaldehyde into acetic acid was accomplished using conventional techniques (e.g. by catalytic oxidizing with atmospheric oxygen in the presence of a mixture of copper and cobalt acetates at 50°-60° C.). The oxidation yields a mixture of acetic acid and acetic anhydride (45:55), with a conversion level between 16 to 18% and a total yield between 94 to 96%.

Oxidation of the acetaldehyde was also accomplished using technical oxygen in the presence of a mixture of manganese, cobalt, nickel and iron salts at 56 to 75° C. and pressure of 0.2 to 0.3 Mpa. The conversion level was 95% and the overall yield is between 92 and 93%.

It will be apparent to those skilled in the art that various modifications and variations can be made in the wheel assembly of the present invention without departing from the spirit or scope of the invention. Thus, it is intended that the present disclosure embrace all reasonable modifications and variations of this invention provided that they come within the scope of any claims and their equivalents.

What is claimed is:

1. A method of preparing phenolic and carbonyl compounds, comprising decomposing hydroperoxides under a positive gas atmosphere and at an elevated temperature in the presence of an anionic surface-active agent of the formula R—OSO₃M, wherein “M” is selected from the group consisting of sodium and potassium and wherein “R” is an alkyl group containing between ten and fourteen carbon atoms.

2 The method of claim 1 wherein said gas atmosphere comprises an inert atmosphere.

3. The method of claim 1 wherein said gas atmosphere is selected from the group consisting of air, nitrogen, and carbon dioxide.

4. The method of claim 1 wherein said elevated temperature is between approximately 80° and approximately 150° C.

5. The method of claim 1 wherein the quantity of said anionic surface-active agent is between approximately 0.01 and approximately 8.0 mass percent.

6. The method of claim 1 wherein said anionic surface-active agent is selected from the group consisting of sodium dodecylsulfate, sodium decylsulfate and sodium tridecylsulfate.

7. The method of claim 1 wherein said hydroperoxide is ethylbenzene hydroperoxide.

8. The method of claim 7 wherein said anionic surface-active agent is sodium dodecylsulfate.

9. The method of claim 8 wherein the quantity of said anionic surface active agent is between approximately 2.0 and approximately 8.0 mass percent of said ethylbenzene hydroperoxide.

10. The method of claim 7 wherein said elevated temperature is between approximately 80° and approximately 150° C.

11. The method of claim 7 further comprising the step of extracting a reaction product from said gas atmosphere by trapping said gas atmosphere and flushing said gas atmosphere through an approximately 20% sulfuric acid solution at approximately 0° C.

12. The method of claim 7 further comprising the step of exposing said gas atmosphere to atmospheric oxygen in the presence of a mixture of copper and cobalt acetates at a temperature between approximately 50° and approximately 60° C.

13. The method of claim 7 further comprising the step of exposing said gas atmosphere to technical oxygen in the presence of a mixture of manganese, cobalt, nickel and iron salts at a temperature between approximately 56° and approximately 75° C.

14. The method of claim 13 wherein the pressure of said technical oxygen is between approximately 0.2 and approximately 0.3 mPa.

15. A method of preparing phenolic and carbonyl compounds, comprising decomposing hydroperoxides under a positive gas atmosphere and at an elevated temperature in the presence of an anionic surface-active agent of the formula R—OP₃Z₂, wherein “Z” is selected from the group consisting of sodium, potassium and alkyl groups containing between ten and fourteen carbons and wherein “R” is an alkyl group containing between ten and fourteen carbon atoms.

16. The method of claim 15 wherein said gas atmosphere comprises an inert atmosphere.

17. The method of claim 15 wherein said gas atmosphere is selected from the group consisting of air, nitrogen, and carbon dioxide.

18. The method of claim 15 wherein said elevated temperature is between approximately 80° and approximately 150° C.

19. The method of claim 15 wherein the quantity of said anionic surface-acting agent is between approximately 0.01 and approximately 0.8 mass percent.

20. The method of claim 15 further comprising the step of extracting a reaction product from said gas atmosphere by trapping said gas atmosphere and flushing said gas atmosphere through an approximately 20% sulfuric acid solution at approximately 0° C.

21. The method of claim 15 further comprising the step of exposing said gas atmosphere to atmospheric oxygen in the presence of a mixture of copper and cobalt acetates at a temperature between approximately 50° and approximately 60° C.

22. The method of claim 15 further comprising the step of exposing said gas atmosphere to technical oxygen in the presence of a mixture of manganese, cobalt, nickel and iron salts at a temperature between approximately 56° and approximately 75° C.

23. The method of claim 22 wherein the pressure of said technical oxygen is between approximately 0.2 and approximately 0.3 mPa.
